(19) United States
(12) Patent Application Publication
(10) Pub. No.: US 2006/0063969 A1
(43) Pub. Date: Mar. 23, 2006

(54) Device for fitting a uterine suspension prosthesis for the treatment of uterine prolapse

(76) Inventor: Edgard Cornier, Paris (FR)

Correspondence Address: BAKER & DANIELS LLP;111 E. WAYNE STREET, SUITE 800, FORT WAYNE, IN, 46802, US

(21) Appl. No.: 11/031,806

(22) Filed: Jan. 7, 2005

(30) Foreign Application Priority Data
Aug. 20, 2004 (FR) 04 51 875

(51) Int. Cl. A61F 2/00

(52) U.S. Cl. 600/37

(57) ABSTRACT

Device for fitting a uterine suspension prosthesis for the treatment of uterine prolapse, characterised in that it is constituted:
on the one hand, by a helical guide catheter (1) produced from a rigid material and provided at its proximal end (P) with a lateral connector (5) of the Luer type capable of being connected to perfusion members, and, at its distal end (D), with a set of sprinkler-rose-type perforations (3), and
on the other hand, by a connecting thread (2) which is capable of sliding in the internal portion of the guide catheter (1) and which is provided at its distal end with a rounded end-piece (7) which has an eye (8) enabling the prosthesis to be slid.

Regardless of its weight in the course of reproductive life or pregnancy, the uterus cannot normally fall into the vagina and is constantly held in a raised position by an assembly of connective fibres which are located behind the peritoneum and which ensure the positioning of the uterus into the manometric enclosure inside of the abdomen.

The connective fibres form two ligamentous bundles, namely a bundle of ligaments extending from the pubis to the sacrum and called the pubic genital sacral ligaments, and a bundle of oblique ligaments directed from the rear to the front and called the pelvic transversal and sagittal hypogastric connective tissues.

Owing to the particular geometry of the peritoneal cavity in the region of its lower portion or neck, the uterus is located in its normal raised position above pressure pincers defined by the posterior portion of the peritoneum (pouch of Douglas) and the anterior portion of the peritoneum (utero-vesical pouch) located between the uterus and the bladder.

In the presence of abdominal efforts, those two pouches close on the conical funnel of the neck of the uterus, so that the uterus is pushed back up and cannot fall into the vagina.

However, it is relatively common for the ligaments that support the uterus, in particular the lateral connective fibres of the pelvic transversal and sagittal hypogastric connective tissues, to slacken and deteriorate with pregnancy or age; such a deterioration may lead to a descent of the uterus or a part of the uterus under the manometric enclosure of the abdomen; it is then expelled from the abdomen and into the vagina every time pressure occurs.

Uterine prolapse causes functional discomfort which may manifest itself only during intense efforts, or which may be permanent, and may therefore have a greatly incapacitating character.

Specialists propose various means of treating uterine prolapse which consist, in particular, in securing the uterus to the vertebral column, closing the pouch of Douglas, or even removing the uterus.

However, none of those means is of an entirely satisfactory nature, as demonstrated by the multiplicity of techniques that have been advocated for a long time now.

The idea on which the invention is based consisted in fitting between the peritoneum and the abdominal wall a prosthesis constituted by a strip of biocompatible fabric, especially of synthetic meshwork, and in suturing that strip by means of non-absorbable threads, on the one hand, to the neck of the uterus by its lower portion and, on the other hand, to the aponeuroses of the abdominal wall by its upper portion, as a replacement for defective connective fibres.

Such a prosthesis enables uterine prolapse to be corrected satisfactorily inasmuch as it puts the mobile uterus back above the pressure pincers of the pouch of Douglas, which it is important to respect, and therefore into the manometric enclosure of the abdomen. The uterus keeps also its movement.

The prosthesis must of course be fitted on the right-hand side and then on the left-hand side of the patient.

In order for the correction to be successful, it is essential that the prostheses are not secured under a high degree of stress since the aim to be achieved is merely to hold the uterus in a position close to that where it is placed by the fragile lateral oblique ligaments and the pubic genital sacral ligaments.

It should be noted that, even though it has to be carried out under general anaesthesia, the fitting of such a prosthesis is a distinctly less serious operation than the operations conventionally carried out to correct uterine prolapse.

The object of the invention is to propose a device permitting the fitting of such a uterine suspension prosthesis.

According to the invention, this device is characterised in that it is constituted, on the one hand, by a helical guide catheter produced from a rigid material and, on the other hand, by a connecting thread of synthetic material capable of sliding in the internal portion of the guide catheter.

The guide catheter is provided at a first of its ends, or its proximal end, with a lateral connector of the Luer type capable of being connected to perfusion members, and, at its second end, or distal end, with a set of sprinkler-rose-type perforations permitting the evacuation to the outside of a perfusion liquid, in particular physiological serum.

The connecting thread for its part projects outside the guide catheter by its proximal end and by its distal end where it is provided with an end-piece, which is rounded, for example, in a spherical shape, and which has or has not an eye enabling the prosthesis to be slid.

The fitting of the prosthesis requires the previous positioning of the guide catheter which must find a passage between the abdominal wall and the peritoneum.

This operation requires the perforation of the patient's abdomen by a cutaneous incision made within the inguinal orifice and then the introduction of the distal end of the guide catheter, which end is equipped with the rounded end-piece, by way of the orifice thus created.

Before introducing the device, the surgeon has to prepare a pericervical incision by natural routes and slightly detach the lower portion of the junction between the bladder and the uterine isthmus.

The practitioner must then move the guide catheter/connecting thread assembly between the peritoneum and the abdominal wall, causing it to progress towards the bottom and towards the front of the abdomen as far as the neck of the uterus, passing behind the bladder.

In the course of the progression of the device in the peritoneal cavity, the Luer-type lateral connector of the guide catheter is constantly connected to a perfusion pouch containing physiological serum.

Consequently, physiological serum is constantly being evacuated to the outside in the immediate vicinity of the distal end equipped with a sprinkler-type rose.

The function of the serum is to enable the spaces to be opened by detaching the peritoneum from the abdominal wall and also from the iliac vessels, the urethra and the bladder, in order to permit access, behind the bladder, to the neck of the uterus to be supported.

The passage of the guide catheter can be effected under visual control (coelioscopy) towards the broad ligament, and the anterior face of the uterus, and then under tactile control behind the bladder and the neck of the uterus at the end of the path.

The guide catheter can also be guided under echographical control owing to the perfusion of physiological serum over the upper portion of the path and then behind the bladder swollen with physiological serum over the second portion of the path.

At the end of the path, the guide catheter is guided tactilely at the anterior face of the uterine isthmus.

When the progression is complete, the distal end of the guide catheter, which end is equipped with the rounded end-piece, is near the surgeon's finger which has been introduced into the vagina and which can then guide the passage thereof and pull on the rounded end-piece in order to cause it to come out and to introduce the prosthesis into the eye thereof, or the prosthesis if it is introduced in a distal eye.

The practitioner can then put the rounded end-piece back in place against the distal end of the guide catheter by pulling on the connecting thread, and then withdraw the guide catheter from the bottom towards the top so that it pulls the prosthesis along with it; or the guide catheter with the prosthesis left in place.

It then only remains for the practitioner to secure the prosthesis, on the one hand, to the neck of the uterus by its lower portion and, on the other hand, to the abdominal aponeuroses by its upper portion.

In order to facilitate the progression of the guide catheter in the internal portion of the peritoneal cavity, the guide catheter preferably has at least a double curvature and comprises from its distal end to its proximal end:
a first portion having the sprinkler-rose-type perforations over a length of at least approximately 1 cm and having a first radius of curvature in a first plane; and
a second portion having a second radius of curvature in a second plane at 45° to the first plane.

The radii of curvature may vary as a function of the size and the structure of the skeleton of the patients; the first radius of curvature is as a general rule approximately 3 cm while the second radius of curvature is as a general rule approximately 3 cm to 5 cm.

According to the invention, the guide catheter may advantageously have a triple curvature and comprise at its proximal end a third portion carrying the lateral connector and having a third radius of curvature in a third plane at 45° to the second plane and at 90° to the first plane.

Bearing in mind that configuration, the guide catheter is in the shape of a helix.

It should be noted that the third radius of curvature, which may also vary as a function of the size and the structure of the skeleton of the patients, is as a general rule approximately 5 cm.

The different portions of the guide catheter may advantageously be separated visually by different colours or marks.

The diameter and the length of the device according to the invention depend for their part largely on the size and corpulence of the patients.

According to another feature of the invention, the guide catheter may be provided with a support handle at its proximal end, in the vicinity of the lateral connector.

The guide catheter may advantageously be produced from a sterilisable thermoformed synthetic material.

It should be noted that it is absolutely necessary to provide for each intervention two symmetrical devices, one of which is adapted to the right-hand side while the other is adapted to the left-hand side of the patient.

The features of the device forming the subject-matter of the invention will be described in greater detail with reference to the appended non-limiting drawings in which:

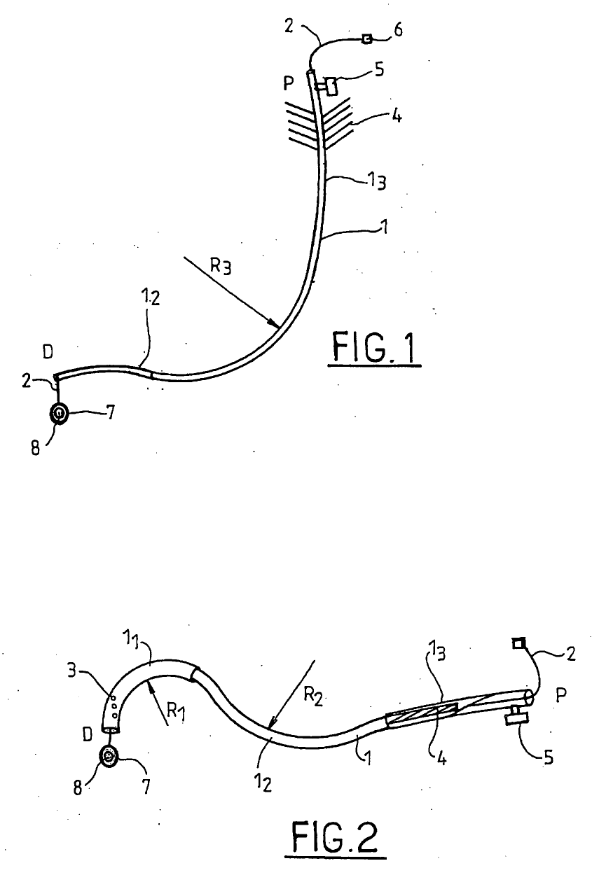

FIG. 1 is a front view of the device;

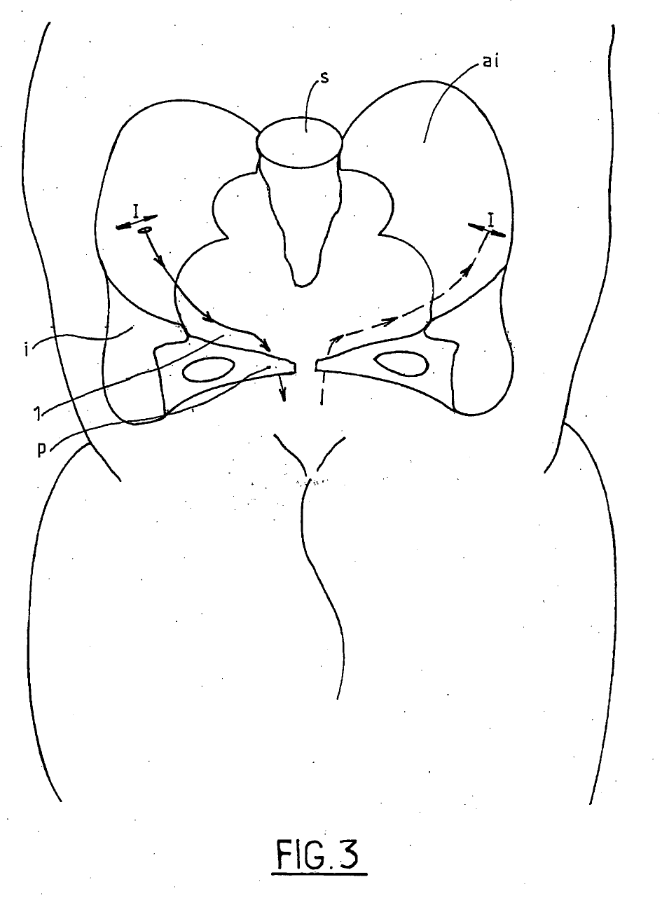

FIG. 2 is a side-face view of the device in a plane offset by 90° relative to the plane of FIG. 1;

FIG. 3 is an explanatory diagram of the fitting of the device.

According to FIGS. 1 and 2, the device is basically constituted by a helical guide catheter 1 produced from a rigid material and by a connecting thread 2 of synthetic material which is capable of sliding in the internal portion of the guide catheter 1 and which projects outside the guide catheter by its two ends.

The assembly thus formed is for single use.

The guide catheter 1 has a triple curvature and comprises from its distal end D to its proximal end P a first portion 1₁ which is not shown in FIG. 1, a second portion 1₂ and a third portion 1₃.

According to FIG. 2, the first portion 1₁ is equipped with a set of sprinkler-rose-type perforations 3 and has a radius of curvature R₁ of approximately 3 cm in a first plane.

The second portion 1₂ has a radius of curvature R₂ of approximately 3 cm to 5 cm in a second plane at 45° to the first plane.

According to FIG. 1, the third portion 1₃ for its part has a radius of curvature R₃ of approximately 5 cm in a third plane at 45° to the second plane and at 90° to the first plane. The guide catheter 1 is thus in the shape of a helix.

These three portions 1₁, 1₂, and 1₃ are separated visually by different colours or marks.

According to FIG. 1, the third portion 1₃ of the guide catheter 1 is equipped at its proximal end, which always remains outside the patient's abdomen, with a handle 4 which is in a single piece and which is secured, especially adhesively bonded or welded, to the guide catheter 1, and with a lateral connector 5 of the Luer type which is to be connected to perfusion members in order to permit the flow in the internal portion of the guide catheter 1 of a perfusion liquid, such as physiological serum, which can be evacuated to the outside by way of the sprinkler-rose-type perforations 3.

According to FIGS. 1 and 2, the connecting thread 2 is equipped at its proximal end P with a button 6 enabling it to be retained on the outside of the guide catheter 1, and at its distal end D with a rounded metal end-piece 7 having an eye 8 enabling a prosthesis constituted by a strip of biocompatible synthetic meshwork, not shown in the Figures, to be slid.

The prosthesis is to be fitted between the peritoneum and the abdominal wall and is to be sutured by non-absorbable threads, on the one hand, to the neck of the uterus by its lower portion and, on the other hand, to the aponeuroses of the abdominal wall by its upper portion, in order to enable uterine prolapse to be treated.

The method of fitting such a prosthesis by means of the above-mentioned device will be described hereinafter with reference to FIG. 3 which is a diagram showing the sacrum s and also the bones of the pelvis of a patient, namely the iliac wing ai, the ischium i as well as the pubis p which are shown in hatched form.

The uterus and the bladder are not shown in this Figure.

The fitting of the prosthesis requires the previous positioning of the guide catheter 1 between the abdominal wall and the peritoneum.

This operation requires the perforation of the patient's abdomen by a cutaneous incision shown diagrammatically by the double arrows I made within the inguinal hole, and then the introduction of the distal end D of the guide catheter, which end is equipped with the rounded end-piece 7, by way of the orifice thus created.

The practitioner must then move the assembly formed by the guide catheter 1 and the connecting thread 2 between the peritoneum and the abdominal wall by causing it to progress towards the bottom and towards the front of the abdomen in accordance with the path represented by a solid line in the left-hand portion of FIG. 3, as far as the neck of the uterus, passing behind the bladder.

At the end of the path, the guide catheter 1 is guided tactilely at the anterior face of the uterine isthmus.

When the progression is complete, the distal end D of the guide catheter 1, which end is equipped with the rounded end-piece 7, is near the surgeon's finger which has been introduced into the vagina and which can then guide the passage thereof and pull on the rounded end-piece in order to cause it to come out and to introduce the prosthesis into the eye 8.

The practitioner can then put the rounded end-piece 7 back in place against the distal end D of the guide catheter 1 by pulling on the connecting thread, and then pull the guide catheter 1 from the bottom towards the top as shown by a dotted line in the right-hand portion of FIG. 3 so that it pulls the prosthesis along with it.

It then only remains for the practitioner to secure the prosthesis, on the one hand, to the neck of the uterus by its lower portion and, on the other hand, to the abdominal aponeuroses by its upper portion.

1. Device for fitting a uterine suspension prosthesis for the treatment of uterine prolapse, the prosthesis being constituted by a strip of biocompatible fabric, especially of synthetic meshwork, which is to be fitted between the peritoneum and the abdominal wall and which is to be sutured by non-absorbable threads, on the one hand, to the neck of the uterus by its lower portion and, on the other hand, to the aponeuroses of the abdominal wall by its upper portion, as a replacement for defective connective fibres,
characterised in that it is constituted:
on the one hand, by a helical guide catheter produced from a rigid material and provided at a first of its ends, or its proximal end, with a lateral connectors of the Luer type capable of being connected to perfusion members, and, at its second end, or distal end, with a set of sprinkler-rose-type perforations permitting the evacuation to the outside of a perfusion liquid, in particular physiological serum, and
on the other hand, by a connecting thread of synthetic material which is capable of sliding in the internal portion of the guide catheter and which projects outside the catheter by its proximal end and by its distal end, which is rounded and which may has an eye enabling the prosthesis to be slid.

2. Device according to claim 1, characterised in that the guide catheter is produced from a sterilisable thermoformed synthetic material.

3. Device according to claim 2, characterised in that the guide catheter is for single use.

4. Device according to claim 1, characterised in that the guide catheter is provided with a support handle at its proximal end, in the vicinity of the lateral connector.

5. Device according to claim 1, characterised in that the guide catheter has at least a double curvature and comprises from its distal end to its proximal end:
a first portion having the sprinkler-rose-type perforations over a length of at least approximately 1 cm and having a first radius of curvature in a first plane; and
a second portion having a second radius of curvature in a second plane at 45° to the first plane.

6. Device according to claim 5, characterised in that the first radius of curvature is approximately 3 cm and the second radius of curvature is approximately 3 cm to 5 cm.

7. Device according to claim 5, characterised in that the guide catheter has a triple curvature and comprises at its proximal end a third portion carrying the lateral connector and, where appropriate, the support handle and having a third radius of curvature in a third plane at 45° to the second plane and at 90° to the first plane.

8. Device according to claim 7, characterised in that the third radius of curvature is approximately 5 cm.

9. Device according to claim 5, characterised in that the different portions of the guide catheter are separated visually by different colours or marks.

10. Device according to claim 2, characterised in that the guide catheter is provided with a support handle at its proximal end, in the vicinity of the lateral connector.

11. Device according to claim 3, characterised in that the guide catheter is provided with a support handle at its proximal end, in the vicinity of the lateral connector.

12. Device according to claim 2, characterised in that the guide catheter has at least a double curvature and comprises from its distal end to its proximal end:
a first portion having the sprinkler-rose-type perforations over a length of at least approximately 1 cm and having a first radius of curvature in a first plane; and
a second portion having a second radius of curvature in a second plane at 45° to the first plane.

13. Device according to claim 3, characterised in that the guide catheter has at least a double curvature and comprises from its distal end to its proximal end:
a first portion having the sprinkler-rose-type perforations over a length of at least approximately 1 cm and having a first radius of curvature in a first plane; and
a second portion having a second radius of curvature in a second plane at 45° to the first plane.

14. Device according to claim 4, characterised in that the guide catheter has at least a double curvature and comprises from its distal end to its proximal end:
a first portion having the sprinkler-rose-type perforations over a length of at least approximately 1 cm and having a first radius of curvature in a first plane; and
a second portion having a second radius of curvature in a second plane at 45° to the first plane.

15. Device according to claim 6, characterised in that the guide catheter has a triple curvature and comprises at its proximal end a third portion carrying the lateral connector and, where appropriate, the support handle and having a third radius of curvature in a third plane at 45° to the second plane and at 90° to the first plane.

16. Device according to claim 6, characterised in that the different portions of the guide catheter are separated visually by different colours or marks.

17. Device according to claim 7, characterised in that the different portions of the guide catheter are separated visually by different colours or marks.

18. Device according to claim 8, characterised in that the different portions of the guide catheter are separated visually by different colours or marks.
